(19) United States
(12) Patent Application Publication
(10) Pub. No.: US 2016/0258945 A1
(43) Pub. Date: Sep. 8, 2016

(54) POINT-OF-CARE IMMUNOSENSING DEVICE FOR MULTI-BIOMARKER DETECTION

(71) Applicant: NORTHEASTERN UNIVERSITY, Boston, MA (US)

(72) Inventors: Asanterabi MALIMA, Lynn, MA (US); Ahmed BUSNAINA, Needham, MA (US)

(21) Appl. No.: 15/027,466

(22) PCT Filed: Oct. 21, 2014
(86) PCT No.: PCT/US14/61542

§ 371 Date: Apr. 6, 2016

Related U.S. Application Data
(60) Provisional application No. 61/893,409, filed on Oct. 21, 2013.

(51) Int. Cl. G01N 33/558; B01L 9/00; B01L 3/00; G01N 33/543

(52) U.S. Cl. CPC G01N 33/558; G01N 33/54386; B01L 9/52; B01L 3/502; G01N 33/54346; B01L 2300/0848; B01L 2300/0887; B01L 2300/0896; B01L 2300/0627; B01L 2200/025; B01L 2200/026; B01L 2300/12

(57) ABSTRACT

An immunosensing device includes a hub mountable to a device for fluid collection, such as a syringe, sample collection container, or vacuum collection container, and a biosensor mounted to the hub. The biosensor includes a biosensor active area in fluid communication with a fluid passage within the hub. The biosensor active area includes nanoelements functionalized with one or more antibodies or antigen binding fragments thereof for contact with a fluid in the fluid passage. The biosensor active area can include multiple regions each including nanoelements functionalized with a different antibody or antigen binding fragment thereof to detect multiple biomarkers. The biosensor, separately or attached to the hub, can be inserted in a biomarker reader for detection of a biomarker in the fluid.

### CROSS-REFERENCE TO RELATED APPLICATIONS

This application claims the benefit under 35 U.S.C. §119(e) of U.S. Provisional Patent Application No. 61/893,409, filed Oct. 21, 2013, entitled Immunosensing Tube/Needle for Multi-Biomarker Detection at the Point-of-care, the disclosure of which is incorporated by reference herein in its entirety.

### STATEMENT REGARDING FEDERALLY SPONSORED RESEARCH OR DEVELOPMENT

N/A

### BACKGROUND

The fields of nanoscience and nanotechnology generally concern the synthesis, fabrication and use of nanoelements and nanostructures at atomic, molecular and supramolecular levels. The nanosize of these elements and structures offers significant potential for research and applications across the scientific disciplines, including materials science, physics, chemistry, computer science, engineering and biology. Biological processes and methods, for example, are expected to be developed based entirely on nanoelements and their assembly into nanostructures. Other applications include developing nanodevices for use in semiconductors, electronics, photonics, optics, materials and medicine.

Recently, assembly of carbon nanotubes and nanoparticles on patterned surfaces has been accomplished using electric fields. A method to assemble nanoelements using a patterned nanosubstrate in a controlled and precise manner using DC electrophoresis is described in U.S. Pat. No. 8,668,978 and WO 2014/143932, the disclosures of which are incorporated by reference herein.

### SUMMARY OF THE INVENTION

An immunosensing device is provided, including a hub, having a fluid passage extending therethrough, and a biosensor mounted to the hub for fluid communication with a fluid in the fluid passage. The hub is mounted to a fluid collection device, such as a syringe, sample collection container, or vacuum collection container. The biosensor includes at least one biosensor active area in fluid communication with the fluid passage within the hub. The biosensor active area includes a plurality of nanoelements disposed on a nanosubstrate. Each nanoelement is functionalized with an antibody or antigen binding fragment thereof. The biosensor active area can include multiple regions each including nanoelements functionalized with a different antibody or antigen binding fragment thereof to detect multiple biomarkers. In this manner, fluid containing one or more biomarkers in the fluid passage is able to contact the at least one biosensor active area of the biosensor. The biosensor, separately or affixed to the hub, can be inserted into a biomarker reader for detection of the one or more biomarkers.

In one aspect, a hub for an immunosensing device is provided comprising a body comprising an external surface, a proximal end and a distal end, a fluid inlet at the distal end, a fluid passage disposed within an interior of the body extending from the fluid inlet at the proximal end; and a biosensor receiving receptacle formed on the body, the biosensor receiving receptacle in fluid communication with the fluid passage within the body.

In another aspect of the hub, the fluid passage comprises a first conduit extending from the fluid inlet to a fluid outlet at the proximal end of the body, and a second fluid conduit extending from the first fluid conduit to the biosensor receiving receptacle and in fluid communication with the first conduit.

In another aspect of the hub, the fluid passage comprises a conduit extending from the fluid inlet to a fluid outlet at the proximal end of the body, and a fluid chamber in fluid communication with the fluid conduit and the biosensor receiving receptacle.

In another aspect of the hub, the biosensor receiving receptacle comprises a recess formed in the external surface of the body.

In another aspect of the hub, the recess comprises a flat area for receiving the biosensor.

In another aspect of the hub, the recess further comprises upstanding walls surrounding the flat area.

In another aspect of the hub, the biosensor receiving receptacle comprises a recess formed within an interior of the body.

In another aspect of the hub, one or more electrical contacts are disposed on the external surface of the body for electrical communication with a biosensor disposed in the biosensor receiving receptacle.

In another aspect of the hub, the one or more electrical contacts are disposed adjacent the biosensor receiving receptacle.

In another aspect of the hub, the one or more electrical contacts are configured for electrical communication with a biomarker reader.

In another aspect of the hub, the body is configured for insertion into a biomarker reader with a biosensor disposed within the biosensor receiving receptacle.

In another aspect of the hub, a needle or a tube is mounted to the fluid inlet of the body.

In another aspect of the hub, the body is configured to mount to a device for fluid collection.

In another aspect of the hub, the body is configured to removably mount to the device for fluid collection.

In another aspect of the hub, the body is configured to mount to a syringe, a sample collection tube, or a vacuum collection tube.

In another aspect of the hub, a biosensor disposed within the biosensor receiving receptacle.

In another aspect of the hub, a covering disposed over the biosensor.

In another aspect of the hub, the covering comprises a removable tab, the biosensor affixed to the tab for removal with the tab from the hub.

In another aspect of the hub, the tab includes an adhesive material on one surface, the biosensor affixed to the tab with the adhesive material.

In another aspect of the hub, the tab includes one or more regions extending beyond edges of the biosensor, and the adhesive material is further affixed to the external surface of the body at the one or more regions extending beyond the edges of the biosensor.

In another aspect of the hub, the biosensor further includes one or more electrical contacts configured for electrical communication with a biomarker reader.

In another aspect of the hub, the biosensor comprises at least one biosensor active area disposed in fluid communication with the fluid passage within the hub, the biosensor active area comprising a plurality of nanoelements disposed on a nanosubstrate, each nanoelement functionalized with an antibody or antigen binding fragment thereof, wherein fluid in the fluid passage within the body contacts the at least one biosensor active area of the biosensor.

In another aspect of the hub, the nanoelements comprise nanoparticles, nanotubes, nanocrystals, dendrimers, or nanowires.

In another aspect of the hub, the nanoelements comprises polystyrene, poly(lactic-co-glycolic acid), carbon nanotubes, single walled carbon nanotubes, organic nanotubes, PSL nanoparticles, silica nanoparticles, or proteins.

In another aspect of the hub, the plurality of nanoelements comprises different types of nanoelements, each type functionalized with different antibody or antigen binding fragment thereof.

In another aspect of the hub, the biosensor active area comprises a plurality of regions, each region including a subset of the nanoelements, the nanoelements within each subset functionalized with a different antibody or antigen binding fragment thereof.

In another aspect, an immunosensing device is provided comprising: a hub mountable to a device for fluid collection, a fluid passage disposed within the hub; and a biosensor mounted to the hub, the biosensor comprising at least one biosensor active area disposed in fluid communication with the fluid passage within the hub, the biosensor active area comprising a plurality of nanoelements disposed on a nanosubstrate, each nanoelement functionalized with an antibody or antigen binding fragment thereof, wherein fluid in the fluid passage contacts the at least one biosensor active area of the biosensor.

In another aspect of the immunosensing device, the biosensor is removably mounted to the hub.

In another aspect of the immunosensing device, the hub further comprises an external surface and the biosensor is mounted to the external surface of the hub.

In another aspect of the immunosensing device, a recess is formed in the external surface of the hub, and the biosensor is disposed within the recess.

In another aspect of the immunosensing device, the recess comprises a flat area for receiving the biosensor.

In another aspect of the immunosensing device, the recess further comprising upstanding walls surrounding the flat area.

In another aspect of the immunosensing device, a covering is disposed over the biosensor.

In another aspect of the immunosensing device, the covering comprises a removable tab, the biosensor affixed to the tab for removal with the tab from the hub.

In another aspect of the immunosensing device, the tab includes an adhesive material on one surface, the biosensor affixed to the tab with the adhesive material.

In another aspect of the immunosensing device, the tab includes one or more regions extending beyond edges of the biosensor, and the adhesive material is further affixed to the hub at the one or more regions extending beyond the edges of the biosensor.

In another aspect of the immunosensing device, the biosensor is affixed to the tab is configured for insertion into a biomarker reader.

In another aspect of the immunosensing device, the biosensor further includes one or more electrical contacts configured for electrical communication with a biomarker reader.

In another aspect of the immunosensing device, the electrical contacts are disposed on a surface of the biosensor.

In another aspect of the immunosensing device, the hub further includes one or more electrical contacts in electrical communication with the biosensor and configured for electrical communication with a biomarker reader.

In another aspect of the immunosensing device, the fluid passage comprises a first conduit extending within the hub, and a second fluid conduit extending from the first fluid conduit to the biosensor active area.

In another aspect of the immunosensing device, the first fluid conduit extends from an inlet to an outlet of the hub, and the second fluid conduit extends from the first fluid conduit at an intermediate location thereof.

In another aspect of the immunosensing device, the fluid passage comprises a conduit extending within the hub and a fluid chamber in fluid communication with the fluid conduit and the biosensor active area.

In another aspect of the immunosensing device, the conduit extends from an inlet to an outlet of the hub, and the fluid chamber is in fluid communication with an intermediate location of the conduit.

In another aspect of the immunosensing device, the hub is configured for insertion into a biomarker reader with the biosensor mounted to the hub.

In another aspect of the immunosensing device, the biomarker reader comprises a device for detecting a biomarker by immunoassay, radio-immunoassay, competitive-binding assay, Western Blot analysis, ELISA assay, immunofluorescence assay, or electrical probe.

In another aspect of the immunosensing device, the device for fluid collection comprises a syringe, a sample collection tube, or a vacuum collection tube.

In another aspect of the immunosensing device, the hub is removably mounted to the device for fluid collection.

In another aspect of the immunosensing device, a hollow tube extends from the hub, the tube comprising a distal end and a proximal end disposed at the hub and in fluid communication with the fluid passage.

In another aspect of the immunosensing device, the hollow tube comprises a hypodermic needle or a sample collection tube.

In another aspect of the immunosensing device, the device for fluid collection comprises a syringe, the hub mountable to the syringe.

In another aspect of the immunosensing device, the hub is removably mounted to the syringe.

In another aspect of the immunosensing device, the nanoelements comprise nanoparticles, nanotubes, nanocrystals, dendrimers, or nanowires.

In another aspect of the immunosensing device, the nanoelements comprise polystyrene, poly(lactic-co-glycolic acid), carbon nanotubes, single walled carbon nanotubes, organic nanotubes, PSL nanoparticles, silica nanoparticles, or proteins.

In another aspect of the immunosensing device, the plurality of nanoelements comprises different types of nanoelements, each type functionalized with different antibody or antigen binding fragment thereof.

In another aspect of the immunosensing device, the biosensor active area comprises a plurality of regions, each region including a subset of the nanoelements, the nanoelements within each subset functionalized with a different antibody or antigen binding fragment thereof.

In another aspect of the immunosensing device, the biosensor further includes a plurality of electrical contacts, and each region is in electrical communication with an associated one of the plurality of electrical contacts.

In another aspect of the immunosensing device, the biosensor comprises a nanosubstrate holder including a terminal end, the biosensor active region disposed at the terminal end.

In another aspect of the immunosensing device, the biosensor further includes one or more electrical contacts disposed on the nanosubstrate holder, and the biosensor active region is electrical communication with the one or more electrical contacts.

In another aspect of the immunosensing device, the biosensor active area is disposed on a chip.

In another aspect of the immunosensing device, the biosensor active area includes nanoelements functionalized to detect a biomarker comprising PSA, CA125, H1N1 virus, HBV antigen, CD46, AZGP1, nucleohistones, or carcinoembryonic antigen.

In another aspect of the immunosensing device, the biosensor active area includes nanoelements functionalized with —NH₂, —CH₂Cl, —CHO (aldehyde), —OSO₂CH₆H₄—CH3, —CHOCH₂ (epoxide), biotin, avidin, or a —COOH group.

In another aspect of the immunosensing device, the biosensor is operative to identify one or more biomarkers present in blood, serum, plasma, urine, saliva, semen, a vaginal secretion, or cerebrospinal fluid.

In another aspect of the immunosensing device, the biosensor active area comprises a nanosubstrate patterned with recesses, the nanoelements disposed within the recesses.

In another aspect of the immunosensing device, the recesses comprise nanotrenches, nanowells, or nanopores.

In another aspect of the immunosensing device, the recesses comprise straight linear depressions, curved linear depressions, intersecting linear depressions, non-intersecting, linear depressions, circular depressions, square depressions, or rectangular depressions.

In another aspect, a method of detecting biomarkers in a biological sample using the immunosensing device is provided, comprising: collecting a biological sample comprising a biomarker in the hub in fluid communication with the biosensor active area of the biosensor; and inserting the biosensor into a biomarker reader, wherein binding of the biomarker to the antibody or the antigen binding fragment thereof results in a detectable signal, thereby detecting the biomarker.

In another aspect of the method, collecting step includes inserting a needle mounted to the hub into a blood vessel of a subject.

In another aspect of the method, in the inserting step, the biomarker reader comprises a device for detecting a biomarker by immunoassay, radio-immunoassay, competitive-binding assay, Western Blot analysis, ELISA assay, immunofluorescence assay, or electrical probe.

In another aspect, a method of detecting biomarkers in a biological sample using the immunosensing device is provided, comprising: collecting a biological sample comprising a biomarker in the hub in fluid communication with the biosensor active area of the biosensor; and determining the presence or absence of the biomarker within the biological sample by immunoassay, radio-immunoassay, competitive-binding assay, Western Blot analysis, ELISA assay, immunofluorescence assay, or electrical probe.

In another aspect, a method of diagnosing a disease or disorder associated with a biomarker in a subject is provided, comprising: collecting a biological sample from the subject using the immunosensing device; contacting the biosensor active area with the biological sample; and determining the presence or absence of the biomarker within the biological sample by immunoassay, radio-immunoassay, competitive-binding assay, Western Blot analysis, ELISA assay, immunofluorescence assay, or electrical probe.

In another aspect, a biosensor for an immunosensing device is provided, comprising: a nanosubstrate holder comprising a biosensor active area and an electrical contact area, the nanosubstrate holder further comprising a planar configuration and a periphery sized to fit within a biosensor receiving receptacle of a hub; the biosensor active area comprising a nanosubstrate patterned with recesses, nanoelements disposed within the recesses, each nanoelement functionalized with an antibody or antigen binding fragment thereof; and a covering, the biosensor affixed to the covering.

In another aspect of the biosensor, the biosensor is affixed to the covering is configured for insertion into a biomarker reader.

In another aspect of the biosensor, the covering comprises a tab including an adhesive material on a surface, the biosensor affixed to the surface of the tab with the adhesive material.

In another aspect of the biosensor, the tab includes one or more regions extending beyond edges of the biosensor, and the adhesive material is further disposed on the one or more regions extending beyond the edges of the biosensor.

In another aspect of the biosensor, the biosensor further includes one or more electrical contacts configured for electrical communication with a biomarker reader.

In another aspect of the biosensor, the recesses comprise nanotrenches, nanowells, or nanopores.

In another aspect of the biosensor, the recesses comprise straight linear depressions, curved linear depressions, intersecting linear depressions, non-intersecting, linear depressions, circular depressions, square depressions, or rectangular depressions.

In another aspect of the biosensor, the nanoelements comprise nanoparticles, nanotubes, nanocrystals, dendrimers, or nanowires.

In another aspect of the biosensor, the nanoelements comprises polystyrene, poly(lactic-co-glycolic acid), carbon nanotubes, single walled carbon nanotubes, organic nanotubes, PSL nanoparticles, silica nanoparticles, or proteins.

In another aspect of the biosensor, the plurality of nanoelements comprises different types of nanoelements, each type functionalized with different antibody or antigen binding fragment thereof.

In another aspect of the biosensor, the biosensor active area comprises a plurality of regions, each region including a subset of the nanoelements, the nanoelements within each subset functionalized with a different antibody or antigen binding fragment thereof.

In another aspect of the biosensor, the biosensor active area includes nanoelements functionalized to detect a biomarker comprising PSA, CA125, H1N1 virus, HBV antigen, CD46, AZGP1, nucleohistones, or carcinoembryonic antigen.

In another aspect of the biosensor, the biosensor active area includes nanoelements functionalized with —NH₂, —CH₂Cl, —CHO (aldehyde), —OSO₂CH₆H₄—CH3, —CHOCH₂ (epoxide), biotin, avidin, or a —COOH group.

In another aspect of the biosensor, the biosensor is operative to identify one or more biomarkers present in blood, serum, plasma, urine, saliva, semen, a vaginal secretion, or cerebrospinal fluid.

### DESCRIPTION OF THE DRAWINGS

The invention will be more fully understood from the following detailed description taken in conjunction with the accompanying drawings in which:

FIG. 1 is a schematic illustration of an embodiment of an immunosensing device in conjunction with a device for fluid collection;

FIG. 2 is an exploded schematic illustration of the immunosensing device of FIG. 1;

FIG. 3 is a schematic side view of the immunosensing device of FIG. 1;

FIG. 4A is a schematic illustration of an embodiment of a biosensor of the immunosensing device;

FIG. 4B is an exploded view of a bioactive sensor area of the biosensor of FIG. 4A;

FIG. 5 is a schematic side view of a further embodiment of an immunosensing device;

FIG. 6A is a schematic illustration of a fluid collection device comprising a syringe with an embodiment of the immunosensing device used for collection of a subject's blood;

FIG. 6B is a schematic illustration of the immunosensing device of FIG. 6A illustration removal of a biosensor along with a covering from a hub;

FIG. 6C is a schematic illustration of the biosensor affixed to the covering for insertion into a biomarker reader;

FIG. 7A is a schematic illustration of a fluid collection device comprising a syringe with a further embodiment of the immunosensing device used for collection of a subject's blood;

FIG. 7B is a schematic illustration of the immunosensing device of FIG. 6A illustration removal of a biosensor along with a hub;

FIG. 7C is a schematic illustration of the biosensor mounted to the hub for insertion into a biomarker reader;

FIG. 8 is a schematic illustration of a further embodiment of an immunosensing device in conjunction with a device for fluid collection; and

FIG. 9 is a schematic illustration of a biosensor active area having multiple regions of nanoelements.

### DETAILED DESCRIPTION OF THE INVENTION

An immunosensing device 10 is provided that allows for the detection of low levels of antigens or disease biomarkers in biological fluids. The immunosensing device is small in size and can be incorporated with a fluid collection device 15, using, for example, a sample collection needle or tube, to allow capture of malignant and infectious disease biomarkers by contact with fluid as the fluid flows through the fluid collection device. The immunosensing device can be used in, for example, hospitals, clinics and physicians' offices, during sample collection.

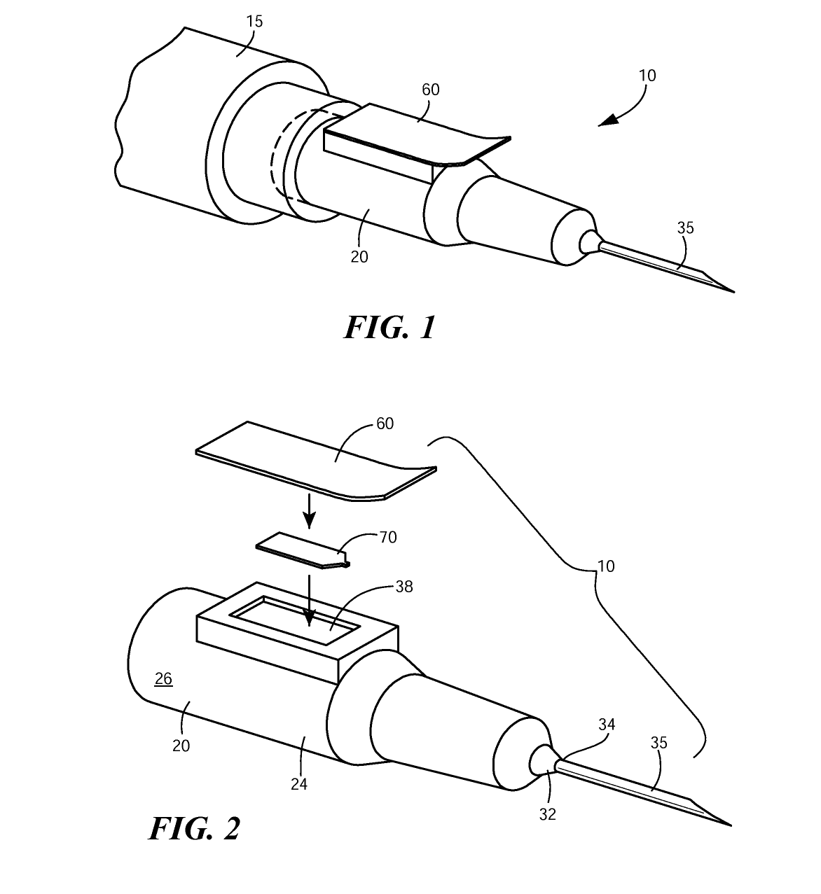

An embodiment of an immunosensing device 10 is described with reference to FIGS. 1-4B. The immunosensing device includes a hub 20, having a fluid passage 22 extending therethrough, and a biosensor 70 mounted to the hub 20 for fluid communication with a fluid in the fluid passage. The biosensor 70 includes at least one biosensor active area 72 in fluid communication with the fluid passage 22 within the hub. The biosensor active area includes a plurality of nanoelements disposed on a nanosubstrate. Each nanoelement is functionalized with an antibody or antigen binding fragment thereof. In this manner, fluid containing a biomarker in the fluid passage is able to contact the at least one biosensor active area of the biosensor.

The hub 20 is mountable to a device for fluid collection 15, such as, without limitation, a syringe, a sample collection container, or a vacuum collection container, indicated schematically in FIG. 1. The hub 20 can be mounted to the fluid collection device in any suitable manner, such as with a latching mechanism, friction fit, or in any other manner known in the art or apparent to one of skill in the art. The hub can be mounted removably or permanently to the fluid collection device. For in vivo applications, the fluid collection device, such as a syringe, can be inserted into a subject for contacting in vivo a biomarker with the biosensor.

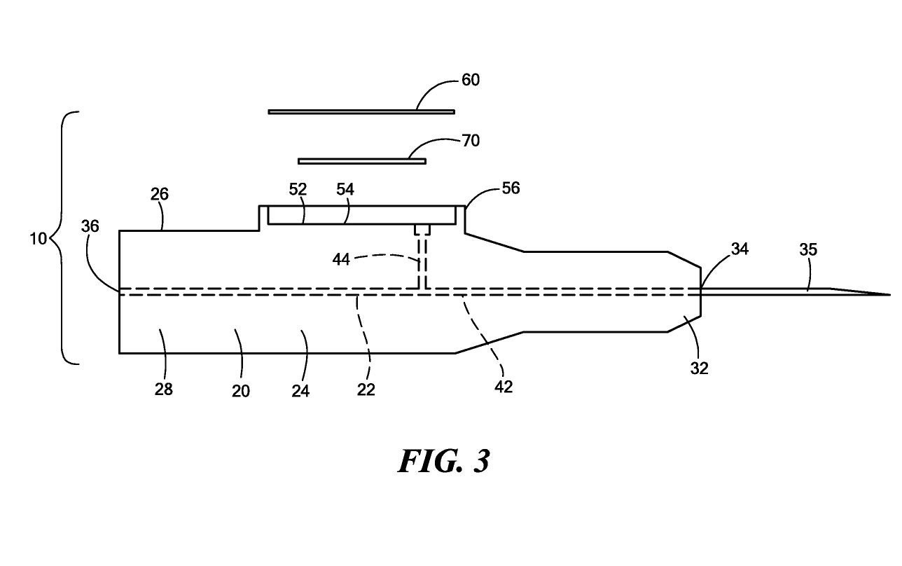

Referring more particularly to FIGS. 2 and 3, in one embodiment, the hub includes a body 24 having an external surface 26, a proximal end 28 and a distal end 32. A fluid inlet 34 is provided at the distal end. The fluid passage 22 is disposed within an interior of the body 24 extending from the fluid inlet 34 at the distal end, generally to a fluid outlet 36 at the proximal end. A tube or needle 35 can be affixed to the inlet of the hub in any suitable manner, removably or permanently, as would be known in the art. A biosensor receiving receptacle 38 is formed on the body. The receptacle is in fluid communication with the fluid passage within the body, such that a fluid in the fluid passage is able to contact the biosensor active area of the biosensor in the receptacle.

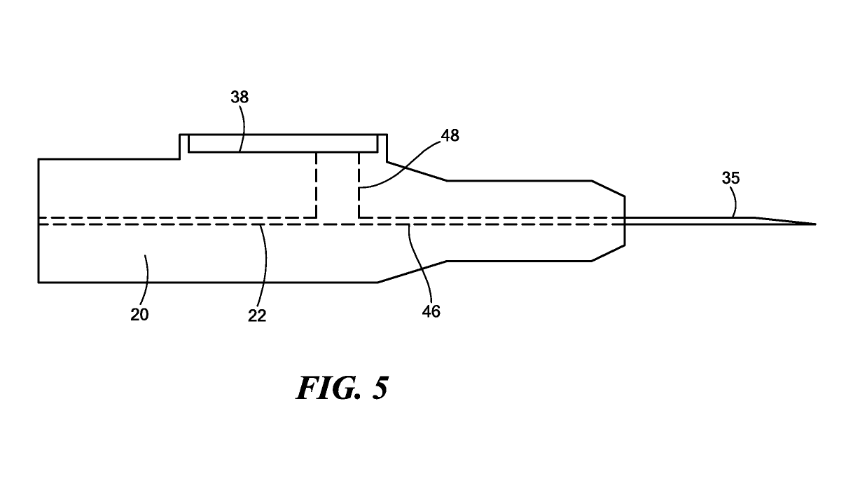

In one embodiment, illustrated schematically in FIG. 3, the fluid passage 22 includes a first conduit 42 extending from the fluid inlet 34 to the fluid outlet 36. A second fluid conduit 44 extends from the first fluid conduit 42 to the biosensor receiving receptacle 38 and in fluid communication with the first conduit. In another embodiment, illustrated schematically in FIG. 5, the fluid passage 22 includes a conduit 46 extending from the fluid inlet to the fluid outlet, and a fluid chamber 48 is provided in fluid communication with the fluid conduit and the biosensor receiving receptacle 38. It will be appreciated that a variety of configurations can be provided to place the biosensing active area in fluid communication with fluid in the body of the hub.

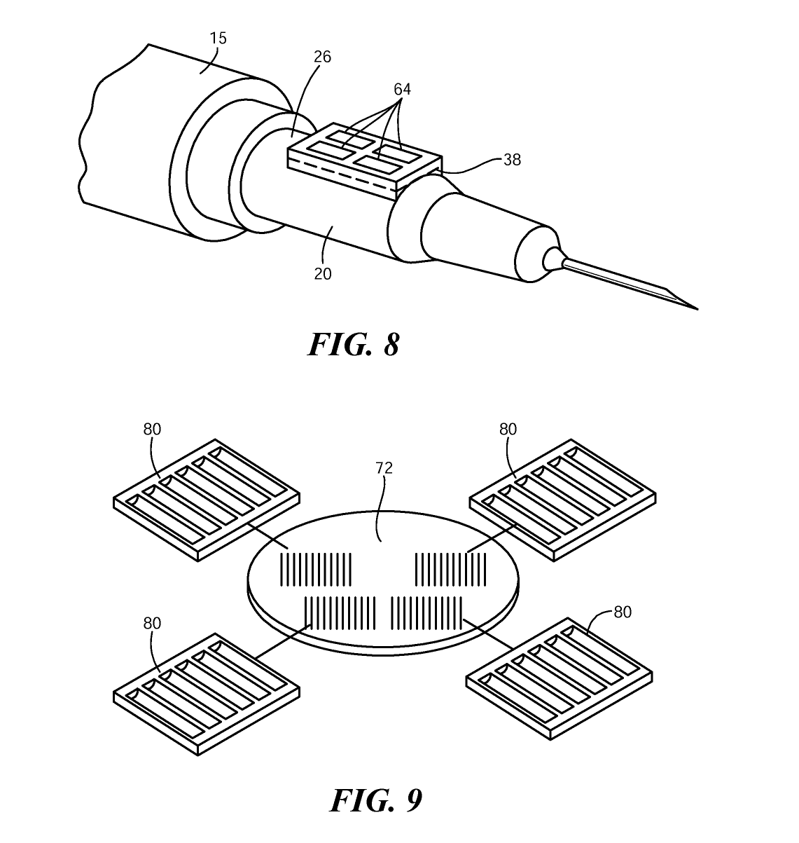

In one embodiment, the biosensor receiving receptacle 38 is provided as a recess 52 formed in the external surface 26 of the body 24 of the hub 20. The recess can have any suitable configuration for receiving a biosensor. In one embodiment, the recess includes a flat area 54 on which the biosensor can be laid. Upstanding walls 56 can be provided surrounding the flat area. In another embodiment, the biosensor receiving receptacle can be formed as a recess within an interior of the body. See FIG. 8. The biosensor can be retained with the receptacle in any suitable manner, such as with an adhesive, a friction fit, a shape conforming to the shape of the receptacle, a cover, or by any other manner, as would be apparent to one of skill in the art.

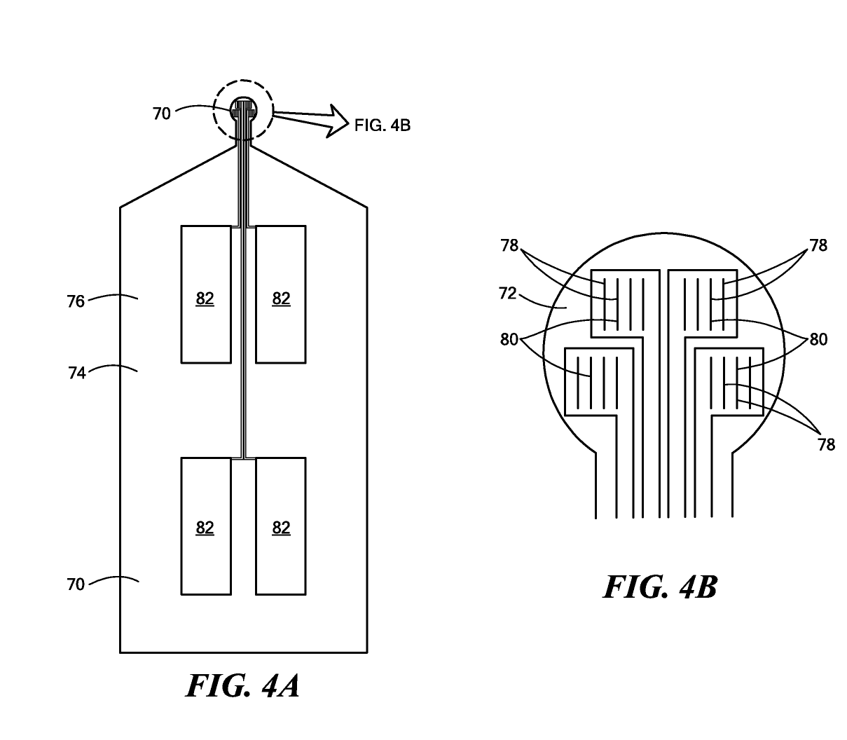

A schematic illustration of a biosensor 70 is illustrated in FIGS. 4A and 4B. In one embodiment, the biosensor includes a nanosubstrate holder 74 including the biosensor active area 72 and an electrical contact area 76. The nanosubstrate holder generally has a planar configuration and a periphery sized to fit within the receptacle 38 of the hub 20. The biosensor active area includes a nanosubstrate patterned with recesses 78, and nanoelements are located within the recesses. Each nanoelement is functionalized with an antibody or antigen binding fragment thereof, as is known in the art. The biosensor active are can include multiple regions 80, and each region can include nanoelements functionalized with a different antibody or antigen binding fragment thereof. In this manner, multiple biomarkers can be detected with a single immunosensing device. In the embodiment shown in FIGS. 4B and 9, four regions are illustrated; however, any suitable number of regions can be provided, such as 1, 2, 3, 5, 6, 7, 8, 9, 10, 12, 15, 20, 25 or more. The electrical contact area 76 includes one or more contact pads 82 each in communication with a region 80 of the biosensor active area. The contact pads can be placed into electrical communication with another device, such as a biomarker reader, or adjacent contacts 64 on the hub.

The recesses 78 on the biosensor can include, for example and without limitation, nanotrenches, nanowells, or nanopores. The recesses can have any suitable geometric configuration, such as, without limitation, straight linear depressions, curved linear depressions, intersecting linear depressions, non-intersecting, linear depressions, circular depressions, square depressions, or rectangular depressions.

The nanoelements can include, for example and without limitation, nanoparticles, nanotubes, nanocrystals, dendrimers, or nanowires. Generally, nanotubes have an aspect ratio of 3 or greater, whereas nanoparticles have an aspect ratio of less than 3 and can include spherical shapes. The nanoelements can be formed from materials such as, without limitation, polystyrene, PLGA polymer (poly(lactic-co-glycolic acid)), carbon nanotubes, single walled carbon nanotubes, organic nanotubes, PSL nanoparticles, silica nanoparticles, or proteins.

In some embodiments, the biosensor active area includes nanoelements functionalized with, for example and without limitation, —NH₂, —CH₂Cl, —CHO (aldehyde), —OSO₂CH₆H₄—CH3, —CHOCH₂ (epoxide), biotin, avidin, or a —COOH group.

In some embodiments, the biosensor active area includes nanoelements functionalized to detect a biomarker comprising, for example and without limitation, PSA, CA125, H1N1 virus, HBV antigen, CD46, AZGP1, nucleohistones, or carcinoembryonic antigen.

Nanoelements for use in the biosensor include, for example, nanocrystals, dendrimers, nanoparticles, nanowires, biological materials, proteins, molecules and organic nanotubes. In certain instances, nanoelements are single walled carbon nanotubes and nanoparticles. In one embodiment, different types of nanoparticles, each type having a different attached antibody or other ligand, can be loaded onto different regions of the sensor using selective voltage activation of the different regions. If the conductive pathways on the nanosubstrate are established such that each region (an area comprising a portion of the nanosubstrate) can be voltage actuated independently, then each region can be loaded with a differently functionalized nanoparticle even though there are no size differences among the different types of nanoparticles. This is accomplished by serially voltage actuating each region in turn while exposed to a suspension of uniquely functionalized nanoparticles (i.e., nanoparticles coated with a different class of antibody, aptamer, or enzyme) for electrophoretic assembly, described further below. In this way a number of different regions of the sensor can be fabricated, each sensitive to a different biological analyte.

The covering 60 can be disposed over the biosensor 70 when the biosensor is installed in the receptacle 38. In one embodiment, the covering is a removable tab formed from a suitable flexible material. The biosensor is affixed to the tab, for example, by an adhesive material on one side of the tab, for removal with the tab from the hub 20. The tab can include one or more regions that extend beyond edges of the biosensor. Adhesive on the extending regions affixes the tab to an external surface of the hub. The tab can be removed from the hub, such as by grasping an end and peeling it off the hub by hand or with a tool. The biosensor, affixed to the tab, is thereby removed along with tab. A portion of the extension regions of the tab can be free of adhesive for ready grasping by a user if desired. In another embodiment, the covering can be a solid member and can be integral with or separable from the body of the hub.

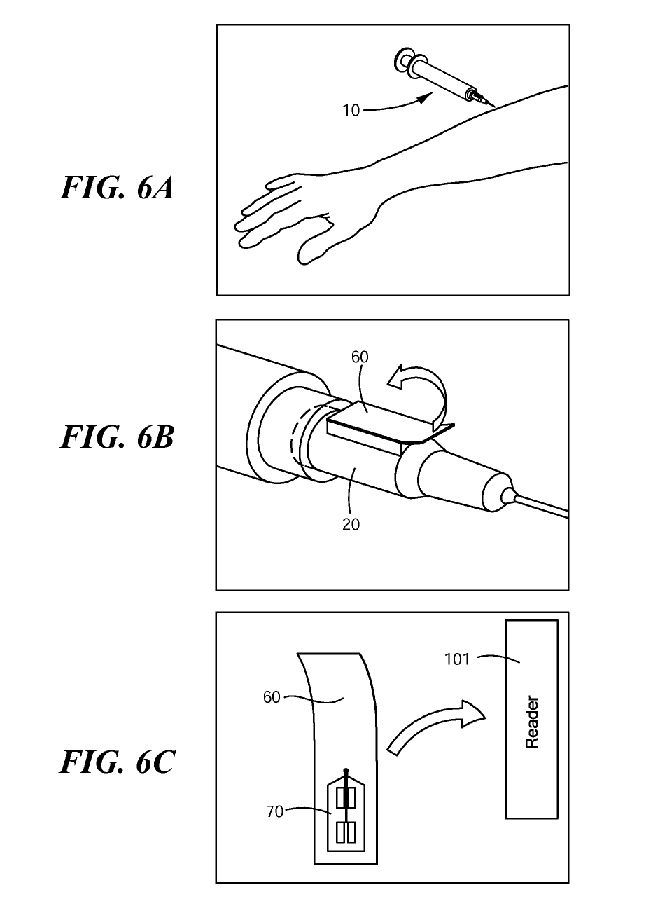

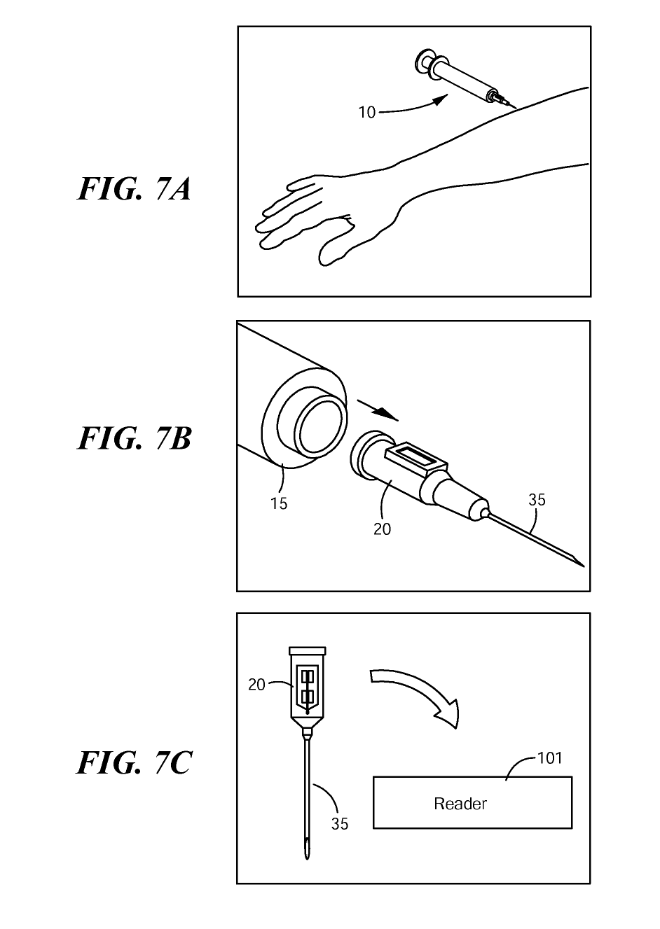

One or more electrical contacts configured for electrical communication with a biomarker reader 101 are provided, on the hub 20 or on the biosensor 70 as contact pads 82. The biomarker reader can be a device for detecting a biomarker by, for example, immunoassay, radio-immunoassay, competitive-binding assay, Western Blot analysis, ELISA assay, immunofluorescence assay, an electrical probe, or any other desired detection method. In one embodiment, the biosensor 70, with contact pads 82, affixed to the covering 60, such as by a tab (described above), can be inserted into a biomarker reader, illustrated schematically in FIGS. 6A-6C. In another embodiment, the hub 20 with the biosensor 70 retained thereto can be inserted into a biomarker reader, illustrated schematically in FIGS. 7A-7C. In one aspect of this embodiment, illustrated schematically in FIG. 8, electrical contacts 64 are provided on the external surface 26 of the body 24 of the hub 20 adjacent the receptacle 38 for electrical communication with the biosensor 70 when disposed in the receptacle and for subsequent electrical communication with the biomarker reader 101. In another aspect of this embodiment, the electrical contacts 82 on the biosensor provide electrical communication with the biomarker reader.

The immunosensing device 10 can be disposed of, for example, in a suitable receptacle for medical or hazardous waste, when its use is complete.

The biosensor can be manufactured in small sizes that can readily fit onto existing configurations of fluid collection devices. In one embodiment, the biosensor active area 72 can be 0.25 mm in diameter, the electrical contact area 76 can be 2 mm×3.5 mm, and each region 80 can be 70 μm×70 μm. In other embodiments, the biosensor active area can have diameters of 0.1 mm, 0.2 mm, 0.3 mm, 0.4 mm, 0.5 mm, 0.75 mm, or 1.0 mm. Greater or lesser dimensions can be provided as well. In other embodiments, the electrical contact area can have length and width dimensions of 0.1 mm, 0.5 mm, 1.0 mm, 2.0 mm, 2.5 mm, 3.0 mm, 3.5 mm, 4.0 mm, 4.5 mm, 5.0 mm, 7.5 mm, and 10.0 mm each. Greater or lesser dimensions can be provided as well. In other embodiments, each region 80 can have length, width, or diameter dimensions of 10 μm, 20 μm, 30 μm, 40 μm, 50 μm, 60 μm, 70 μm, 80 μm, 90 μm, 100 μm, 150 μm, 200 μm. Greater or lesser dimensions can be provided as well.

The immunosensing device can be manufactured in any suitable manner. For example, the hub can be manufactured by, for example and without limitation, injection molding. Any suitable material can be used, such as without limitation a plastic or polymer material. A biosensor can be incorporated into the hub at the time of manufacture or can be fabricated separately and disposed on or in the hub subsequently, for example, by a user of the immunosensing device.

The biosensor for use in the immunosensing device can be manufactured in any suitable manner. Exemplary biosensors can be made by, for example, sequentially depositing a sacrificial layer, a conductive layer, and an insulating layer onto the substrate layer, and then removing selected areas of the insulating layer by lithography. The removed areas form recesses, such as nanotrenches or nanowells. A plurality of nanoelements are then assembled within the recesses, for example, by DC electrophoresis. Exemplary manufacturing processes are described in, for example, U.S. Pat. No. 8,668,978 and WO 2014/143931, incorporated by reference herein.

In one example, the base layer of a nanosubstrate is the substrate layer. The substrate layer typically extends the length and width of the nanosubstrate and provides structural rigidity. The substrate layer supports the other layers which are added to one surface of the substrate layer. The thickness of the substrate layer is not critical for most applications, and can range, for example from about 100 nm to about several cm or more. A wide variety of non-conducting materials can be used for the substrate layer of a nanosubstrate. Silicon wafers, for example, are capable of being used as a substrate layer. A particular material is silicon dioxide (SiO₂, also referred to as silicon oxide). Other suitable materials include organic or inorganic insulating materials, e.g., non-conducting oxides. Additional materials include silicon, quartz, a glass wafer, GaSb, SOI, GaAs, GaP, GaN, Ge, InP, ZnO, SiO, CdSe, CdTe, ZnS, ZnSe, ZnTe, and Al₂O₃. The substrate layer is electrically insulating so that it does not provide current leak pathways that might alter the intended electric field distribution for nanoelement assembly. The substrate layer should be structurally rigid so that the nanoscale structural features of the insulating layer are stably preserved with respect to one another. In some instances, the substrate layer of a nanosubstrate has a smooth surface topology. The substrate layer can be formed by available methods for cutting, polishing, molding, or polymerizing suitable materials, as is well known in the art. The substrate layer can have any desired shape or thickness, but in particular instances, it is a thin sheet or film having an approximately flat surface on at least one side upon which the other layers can be deposited. The two-dimensional shape outlined by the surface of the substrate layer that receives the additional layers of the nanosubstrate can be, for example, circular, rectangular, square, irregular, or another shape.

In embodiments where the nanosubstrate does not include a sacrificial layer, a first substrate layer can be used during the fabrication process, which is ultimately removed from a second substrate layer (onto which the conducting layer and the insulating layer are deposited). In such instances, the first substrate layer can be a material described above, such as a silicon wafer. The second substrate layer can be any material, such as a polymer described herein. Nonlimiting exemplary materials for the second substrate layer include photoresist (e.g., SU-8), PDMS and Parylene.

In one embodiment, the substrate layer has three additional layers adsorbed onto one of its surfaces. These layers can be deposited by any method that provides a generally homogeneous, thin layer with good molecular contact and adhesion to adjacent layers. For example, chemical vapor deposition and physical vapor deposition are suitable methods for depositing metals. One nonlimiting method for depositing metals is sputtering. Polymers such as PMMA can be deposited in the liquid state, for example, by spin coating. If appropriate, suitable methods can be employed to harden the polymer layer, e.g., exposure to heat, light, or chemicals.

The sacrificial layer is a thin film that allows a lift off process to be performed, so that a preformed biochip precursor based on a second substrate (e.g., SU-8) can be removed from a first substrate (e.g., silicon) and subsequently processed to form a functioning biochip. The sacrificial layer can be made of chromium, for example, or another material suitable for a lift off process. The sacrificial layer can be any thickness compatible with its role in allowing lift off of the lithographically patterned second substrate from the first substrate, such as about 1 μm thickness. In embodiments where the sacrificial layer is removed during the fabrication of a nanosubstrate, the sacrificial layer can be removed by known methods, such as etching (e.g., isotropic etching, wet etching).

The conductive layer establishes a uniform electric field that drives the assembly of nanoelements on the nanosubstrate. Suitable materials for the conductive layer include any highly conductive metals or metal oxides. Nonlimiting, exemplary conductive materials include carbon ink, silver ink, Ag/AgCl ink, copper, nickel, tin, gold, aluminum, or platinum. The conductive layer can be deposited using any known method, such as metal deposition (such as sputtering (e.g., magnetron sputtering), sputter deposition, vapor deposition, thermal spray coating, and ion beam techniques), electrodeposition coating, etching, and self-assembly. The thickness of the conductive layer is chosen in order to minimize resistance, provide adequate conductivity and a uniform electric field, and good adhesion to adjacent layers. For example, the thickness can be in the range of about 50 nm to about 100 nm. An alternative to using a gold or other metallic conductive layer is to use a conductive polymer such as polyanaline. In this way, a completely biocompatible device can be made, such as a sensor or array for implantation in an animal body or for analysis of metal sensitive proteins in vitro or in vivo.

An insulating layer is added onto the conductive layer, followed by lithography (e.g., electron-beam lithography) to make nanoscale trenches (either linear or curved) or nanoscale wells. Nonlimiting, exemplary materials for the insulating layer include PMMA [poly(methyl methacrylate)], ZEP-520A, APEX-E SAL-601, SNR-200, UVN2, UVN30, UV5, and NEB.

Following exposure, a portion of the insulating layer is removed (e.g., PMMA film is dissolved in acetone) and, after rinsing in deionized water, the gold surface is exposed for the electrical connection. The plain conductive gold surface ensures that a uniform potential is applied underneath the patterned insulating layer, and the electric field distribution is controlled by the patterned insulating PMMA film. The patterns of nanotrenches or nanowells formed by lithography leave desired areas of the conductive layer exposed to the fluid environment containing dispersed nanoelements and determine the pattern of alignment and assembly of nanoelements during electrophoresis. This design has the advantage of achieving consistent assembly over a large area wherever the potential and geometric design of nanotrenches or nanowells are the same. Nanotrenches or nanowells are at least about 20 nm in width or diameter. In certain embodiments, the nanotrenches or nanowells are also less than about 100000 nm, about 10000 nm, about 1000 nm, or about 500 nm in width or diameter. Nanotrenches can be at least about 50 nm in length, and in certain embodiments can be at least about 100 nm, about 500 nm, about 1000 nm, about 10000 nm, about 100000 nm or more in length.

In forming the insulating layer, an electrically insulating material is deposited directly onto the conductive layer in a liquid state. A monomeric material can be used to coat the conductive layer, followed by polymerization of the monomer by any of a variety of methods. These methods include, but are not limited to, free radical polymerization, photopolymerization, anionic polymerization and cationic polymerization. Polymeric liquids also can be used to insulate the conductive layer, for example, by thermal treatment or photocuring. Any insulating material compatible with a suitable lithography process can be used. One particular material is PMMA. The thickness of the insulating layer is sufficient to provide good electrical insulation, so as not to attract charged nanoelements to unintended areas of the nanosubstrate, and will depend on the dielectric properties of the material. The thickness is also compatible with complete removal by lithography to expose the conducting layer. For example, the range of thickness for the insulating layer can be about 80 nm to about 150 nm.

Any lithographic process capable of selectively removing desired areas of the insulating layer and exposing the conductive layer beneath can be used. Nonlimiting processes include electron-beam, ion-beam, ultraviolet, extreme ultraviolet or soft lithographies. Comparable methods such as holographic, nanoimprint, immersion or interference lithographies can also be used. Generally, a nanosubstrate patterned by one of the above methods features surface depressions or recesses, usually in the form of nanotrenches or nanowells, resulting in exposure of the underlying conductive layer.

A variety of patterns can be created by lithography of the insulating layer of a nanosubstrate of the invention, depending on the geometry of the nanoelements being assembled and the desired end product. Nanotrenches are linear depressions that can be straight or curved as well as intersecting or non-intersecting. Nanowells are approximately circular, square, or rectangular depressions. The nanotrenches or nanowells on a given nanosubstrate can have similar dimensions or different dimensions. The assembly by DC electrophoresis of nanoelements on a nanosubstrate can be used regardless of which type of pattern is present in the insulating layer.

Lithographically constructed patterns formed on individual nanosubstrates can be combined to make larger patterns. There is in principle no upper limit to the pattern size, or to the width or length of assembled nanoelements that can be made.

Methods for directing the assembly of nanoelements such as carbon nanotubes and nanoparticles on structured substrates can use DC electrophoresis. The method employs a nanosubstrate as described above to generate a nanopatterned electric field in a liquid suspension containing charged nanoelements. The field is established by connecting a DC voltage source to the nanosubstrate as one electrode and to a second electrode. Optionally, an ammeter can be used to track current flow during assembly. The field causes the movement by electrophoresis of the nanoelements toward the nanosubstrate. Conditions can be selected such that the nanoelements carry a negative charge, in which case they will migrate toward the anode during electrophoresis. If the conductive layer of the nanosubstrate is chosen as the anode, then nanoelements from the liquid suspension will accumulate and form an assembly on the conductive layer inside the nanotrenches or nanowells formed by the insulating layer. If desired, the assembly can be exposed or removed from the nanosubstrate by eliminating the insulating layer (e.g., dissolving a PMMA layer with acetone and rinsing with deionized water).

Nanoelements can be made of any suitable known material. Nonlimiting materials include, e.g., polystyrene and PLGA polymer (poly(lactic-co-glycolic acid). Nanoelements including carbon nanotubes and PSL or silica nanoparticles typically have a net charge at pH values above or below their isoelectric points. At a pH above the isoelectric point, nanoelements will be negatively charged. Therefore, in some embodiments, during manufacture, the pH of the nanoelement suspension is adjusted to above the isoelectric point of the nanoelements, and the conductive layer of the nanosubstrate is used as the anode and will attract the particles when a voltage is applied. Alternatively, the pH of the suspension can be set to below the isoelectric point of the nanoelements, and the conductive layer of the nanosubstrate is used as the cathode.

Regardless of the polarity of the conductive layer of the nanosubstrate during electrophoresis, the other electrode (second electrode) is placed into the suspension at some known distance from the nanosubstrate. For example, if the conductive layer of the nanosubstrate is the anode, then the cathode will be present in the nanoelement liquid suspension, for example at a distance of about 1 cm removed from the nanosubstrate. A uniform electric field is provided between the conductive layer of the nanosubstrate and the second electrode. This can be accomplished by assuring that the other electrode is equidistant from the nanosubstrate over the full area of the nanosubstrate. For example, if the nanosubstrate is a planar rectangle, then the second electrode should also be planar and arranged parallel to the entire exposed area of the conductive layer of the nanosubstrate. The second electrode can be fabricated of any appropriate conductive material, such as the same material as the conductive layer of the nanosubstrate (e.g., a gold film on a substrate).

In some instances, the nanoelement suspension used as a feed source for assembly can be an aqueous suspension. In other instances, other liquids such as alcohols or other polar solvents can be used, as can mixtures of water and other aqueous solvents. The suspension can contain a sufficient ionic strength such that some level of charge screening occurs at charged positions on the nanoelements. Otherwise, aggregation or nonspecific binding of the nanoelements can occur, which would prevent their orderly assembly at the nanosubstrate. In one embodiment, a small amount of ammonium hydroxide solution, resulting in a final concentration in the range of about 0.5 μM to about 1 μm is added to a deionized water suspension of nanoelements. This provides both the requisite ionic strength and sets the pH of the solution to the desired range of about 7 to about 8.

The conductive layer of the nanosubstrate is connected to a regulated DC power supply, such as one providing constant voltage adjustable in the range of about 1 V/cm to about 5 V/cm between the electrodes. Electrical connection with the connective layer of the nanosubstrate can be established by a variety of conventional techniques. One suitable method is to leave a portion of the conductive layer exposed (i.e., without any overlaying insulating layer) at an edge of the nanosubstrate so that electrical contact with the conductive film can be made. In general, the stronger the electric field, the more rapid assembly will take place. A threshold voltage may exist below which no assembly occurs, and too high a voltage will lead to breakdown of the conductive layer with subsequent disruption of assembly. Smaller dimensions of the nanotrenches or nanowells generally requires a higher voltage to drive assembly. An appropriate voltage for a given set of conditions is readily determined by trial.

The nanoelements can be comprised of two or more different size classes. A nanosubstrate is fabricated with nanotrenches or nanopores of two or more different widths. Nanoelements of different size classes are assembled on the nanosubstrate in decreasing order of size. In each cycle, nanoelements of a size class are assembled in a nanotrench or nanopore of similar or slightly greater size as the average width or diameter of the nanoelements. In that way, each size class of nanoelements can be targeted to one or more specific nanotrenches or nanopores. In certain embodiments, nanoelements belonging to different size classes can be differentially functionalized, resulting in spatially distributed chemical groups that can be employed, for example, as an array or biosensor. For example, each of the nanoparticle classes has been functionalized and bound to a different type of antibody or fragment of an antibody. When an antigen is present which binds to one of the antibody types, but not the other, a specific signal is generated that indicates the presence and identity of the antigen. For example, a second antibody that binds to the antigen and possesses a bound label, such as a fluorescent tag or an enzyme, can be used to detect antigens bound to the nanosubstrate.

Antibodies can be attached to nanoparticles described herein using standard methods. For example, nanoparticles can be functionalized on their surface with —NH₂, —CH₂Cl, —CHO (aldehyde), —OSO₂CH₆H₄—CH3, —CHOCH₂ (epoxide), biotin, and avidin.

In one exemplary method for attaching an antibody to a nanoparticle, first, a polystyrene bead is functionalized on its surface with a —COOH group. Next, an antibody is incubated with the functionalized nanoparticle suspended in a saline buffered solution, such as overnight. Unbound antibody can then be removed from the bead suspension by ultracentrifugation for, e.g., 15 minutes at 12.times.1000 rpm.

Any antibody, or antigen-binding portion thereof, can be attached to a nanoparticle described herein. Exemplary antibodies include, without limitation, mAb-2C5 (Iakoubov et al. (1997) Oncol. Res. 9:439-446), mAb to carcinoembriogenic antigen (Hammarstrom (1999) Semin. Cancer Biol. 2:67-81), or antibodies that bind to biomarkers such as PSA (prostate specific antigen), CA125 (ovarian cancer antigen), H1N1 virus, HBV antigen (hepatitis B virus), CD46 (membrane cofactor protein to malignant neoplasm of prostate, bacterial infections, astrocytoma, glioblastoma, gonorrhea), and AZGP1 (alpha-2-glycoprotein to cardiac hypertrophy, E. coli infection to Central Nervous System), as well as antibodies that bind to targets related to cardiovascular disease, such as cardiac myosin, cardiac troponin I, or C-reactive protein. Antigen-specific binding portions of antibodies can also be used, such as Fab, Fab′2, and Fv, and the antibodies may be genetically engineered or naturally produced using known methods. Alternatively, other binding agents specific for the disease markers may be used, such as enzymes or nucleic acid or peptide aptamers, which have specificity, through their binding activity, for various biomolecules.

### Methods of Using Biosensor Devices for Diagnosis

The biosensor devices described herein can be used to identify a subject having, or at risk of developing, a disease or disorder. Certain methods include obtaining a biological sample from a subject and a sample from a control subject not having, or not at risk of developing, the disease or disorder, and contacting a biosensor device with the biological samples. The biological sample can be, e.g., urine, blood, serum, plasma, saliva, semen, a vaginal secretion, or cerebrospinal fluid. In some instances, the biological sample is a plasma sample.

In other methods, a biosensor device described herein can be inserted into a subject, and the biosensor device contacts one or more biomarkers in vivo. For example, a biosensor device, such as a biosensor device attached to a hypodermic syringe, can be inserted into the body of a subject, such as a blood vessel, of the subject. The biosensor device can then be removed from the subject and the level of one or more biomarkers can be detected as described herein. In particular instances, a biosensor device can be used to detect the level of a plurality of biomarkers, such as 2, 3, 4, 5, 6, 7, 8, 9, 10, 20, 30, 40, 50, 100, 250, 500, 750, 1000 biomarkers, or more.

Any known biomarker can be used to identify a subject having, or at risk of developing, a disease or disorder. If the level of one or more of these biomarkers is different relative to the control level, the subject can be classified as having, or at risk of developing, a disease or disorder associated with the biomarker.

For example, the level of one or more of the following biomarkers can be measured: PSA (prostate specific antigen), CA125 (ovarian cancer antigen), H1N1 virus, HBV antigen (hepatitis B virus), CD46 (membrane cofactor protein to malignant neoplasm of prostate, bacterial infections, astrocytoma, glioblastoma, gonorrhea), and AZGP1 (alpha-2-glycoprotein to cardiac hypertrophy, E. coli infection to Central Nervous System). Other biomarkers are described in, e.g., U.S. Pat. Nos. 7,666,583 and 7,537,938.

Yet other biomarkers are nucleohistones (NHS) and carcinoembryonic antigen (CEA), which are two of the many biomarkers that are pathologically indicated in diseased or cancerous condition. NHS is found in diseases such as Systemic Lupus Erythematosus (SLE), and CEA is found in various cancerous conditions such as colorectal, gastric, pancreatic, lung and breast carcinomas. NHS are classic biomarkers that are released by most carcinomas and are not associated with a particular type of cancer.

Once a subject is identified as having, or at risk of developing, a disease or disorder, the subject can be treated with an appropriate therapy for the condition.

Methods of Using Biosensors

The biosensors described herein can be used to detect the level of a biomarker, such as a polypeptide or other antigen, in a biological sample from a subject. Exemplary biological fluids include, but are not limited to, blood, plasma, lacrimal secretions, saliva, seminal fluid, vaginal secretion, sweat, mucous, or urine. In some instances, the biosensor is contacted with the biological fluid and then post-processed for the detection of binding of a biomarker to an antibody on a nanoelement on the biosensor.

The detection of a biomarker can be performed using methods known in the art. Such assay methods include, but are not limited to, immunoassays, radio-immunoassays, competitive-binding assays, Western Blot analysis, ELISA assays, and immunofluorescence assays.

In certain instances, an elevated level of a biomarker relative to a control indicates a risk of disease or disorder. In other instances, a reduced level of a biomarker relative to a control indicates a risk of disease or disorder.

In some instances, a biomarker is detected after separation from a biological sample. Separation techniques include, but are not limited to, column chromatography, filtration, ultrafiltration, salt precipitation, solvent precipitation, solvent extraction, distillation, immunoprecipitation, SDS-polyacrylamide gel electrophoresis, isoelectric point electrophoresis, dialysis, and recrystallization.

For chromatography, affinity chromatography, ion-exchange chromatography, hydrophobic chromatography, gel filtration, reverse phase chromatography, adsorption chromatography, and such may be used (see, e.g., Strategies for Protein Purification and Characterization: A Laboratory Course Manual. Ed, Daniel R. Marshak et al., Cold Spring Harbor Laboratory Press (1996)). These chromatography procedures can also be liquid chromatography, such as HPLC and FPLC.

In some instances, the presence of biomarkers in a biological sample can be measured by optionally modifying or partially degrading the proteins in a biological sample, for example, by treating the biological sample with an appropriate protein modification enzyme before separation. Such a modification or partial degradation can be utilized when, for example, the proteins in a biological sample are not easily separated. Such protein modification enzymes include, for example, trypsin, chymotrypsin, lysylendopeptidase, protein kinase, and glucosidase.

In certain instances, multidimensional separation techniques, such as tryptic peptide fractionation using reversed phase and ion exchange LC, or protein pre-fractionation methods, like ion exchange, size exclusion, hydrophobic interaction and various affinity methods, can be used (Martosella, et al., J. Proteome Res. (2005) 4:1522-1537). One nonlimiting example of a pre-fractionation method includes removing high abundance proteins to reduce the dynamic range of protein levels in biological fluids to better match that of the analytical platform. variety of depletion methods for specific removal of high abundance proteins from bodily fluids can be used (see, e.g., Govorukhina, et al., J. Chromatogr. A (2003) 1009:171-178). A nonlimiting example is the multiple affinity removal system (MARS, Agilent, Palo Alto, Calif.), which utilizes an affinity column. This column can deplete albumin, IgG, IgA, transferrin, haptoglobin and antitrypsin in human plasma (Ogata, et al., J. Proteome Res. (2005) 4:837-845; Bjorhall, et al., Proteomics (2005) 5:307-317). The MARS column can deplete these proteins from 30-40 μl of plasma at a time and can be regenerated up to 200 times.

Another separation technique that can be used in the methods disclosed herein involves using a combination of three lectins in the form of a multi lectin column (M-LAC). This affinity column can capture and enrich fractions, e.g., glycoprotein fractions, in plasma. In some instances, fractions can be subjected to LC-MS after tryptic digestion (Yang, et al., J. Chromategr. A (2004) 1053:79-88).

### Diseases/Disorders

The biosensor devices described herein can be used to diagnose many types of diseases or disorders.

In particular instances, a biosensor device is used to diagnose hyperproliferative, hyperplastic, metaplastic, dysplastic, or pre-neoplastic diseases or disorders.

By “hyperproliferative disease or disorder” is meant a neoplastic cell growth or proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and cancer. Additional nonlimiting examples of hyperproliferative diseases, disorders, and/or conditions include neoplasms, whether benign or malignant, located in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract.

As used herein, the term “tumor” or “tumor tissue” refers to an abnormal mass of tissue that results from excessive cell division. A tumor or tumor tissue comprises “tumor cells”, which are neoplastic cells with abnormal growth properties and no useful bodily function. Tumors, tumor tissue, and tumor cells may be benign or malignant. A tumor or tumor tissue can also comprise “tumor-associated non-tumor cells”, such as vascular cells that form blood vessels to supply the tumor or tumor tissue. Non-tumor cells can be induced to replicate and develop by tumor cells, for example, induced to undergo angiogenesis within or surrounding a tumor or tumor tissue.

As used herein, the term “malignancy” refers to a non-benign tumor or a cancer. As used herein, the term “cancer” means a type of hyperproliferative disease that includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include squamous cell cancer (e.g., epithelial squamous cell cancer), lung cancer (including small-cell lung cancer, non-small cell lung cancer, adenocarcinoma of the lung and squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer, uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to, Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Fibrosarcoma, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, and Wilm's Tumor.

The methods described herein can also be used to diagnose premalignant conditions, e.g., to prevent progression to a neoplastic or malignant state including, but not limited to, those disorders described above. The methods described herein can further be used to diagnose hyperplastic disorders. Hyperplasia is a form of controlled cell proliferation, involving an increase in cell number in a tissue or organ, without significant alteration in structure or function. Hyperplastic disorders include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

The methods described herein can also be used to diagnose metaplastic disorders. Metaplasia is a form of controlled cell growth in which one type of adult or fully differentiated cell substitutes for another type of adult cell. Metaplastic disorders include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

The methods described herein can also be used to diagnose dysplastic disorders. Dysplasia can be a forerunner of cancer and is found mainly in the epithelia. Dysplasia is a disorderly form of non-neoplastic cell growth, involving a loss in individual cell uniformity and in the architectural orientation of cells. Dysplastic cells can have abnormally large, deeply stained nuclei, and exhibit pleomorphism. Dysplasia can occur, e.g., in areas of chronic irritation or inflammation. Dysplastic disorders include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of the jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders that can be diagnosed by the methods described herein include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

The term “biological sample” refers to a material obtained from an organism or from components (e.g., cells) of an organism. The sample may be of any biological tissue or fluid, for example, a sample derived from a patient. Such samples include, but are not limited to, blood, blood cells (e.g., white cells), plasma, tissue or fine needle biopsy samples, urine, peritoneal fluid, and pleural fluid, or cells there from. Biological samples may also include sections of tissues such as frozen sections taken for histological purposes.

The term “biomarker” of a disease or condition refers to a gene or a gene product that is up- or down-regulated in a biological sample of a subject having the disease or condition relative to a biological sample from like tissue derivation, which gene or gene product is sufficiently specific to the disease or condition that it can be used, optionally with other genes or gene products, to identify or detect the disease or condition. Generally, a biomarker is a gene or a gene product that is characteristic of the disease or condition.

The term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab, F(ab′).sub.2, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda.

A “subject” is a mammal, e.g., a human, mouse, rat, guinea pig, dog, cat, horse, cow, pig, or non-human primate, such as a monkey, chimpanzee, baboon or rhesus.

It will be appreciated that the various features of the embodiments described herein can be combined in a variety of ways. For example, a feature described in conjunction with one embodiment may be included in another embodiment even if not explicitly described in conjunction with that embodiment. The various components of the immunosensing device can be manufactured from a number of suitable materials, as would be apparent to one of skill in the art.

The present invention has been described with reference to the preferred embodiments. It is to be understood that the invention is not limited to the exact details of construction, operation, exact materials or embodiments shown and described, as obvious modifications and equivalents will be apparent to one skilled in the art. It is believed that many modifications and alterations to the embodiments disclosed will readily suggest themselves to those skilled in the art upon reading and understanding the detailed description of the invention. It is intended to include all such modifications and alterations insofar as they come within the scope of the present invention.

1. A hub for an immunosensing device, the hub comprising:
a body comprising an external surface, a proximal end and a distal end, a fluid inlet at the distal end, a fluid passage disposed within an interior of the body extending from the fluid inlet at the is distal end; and
a biosensor receiving receptacle formed on the body, the biosensor receiving receptacle comprising a recess formed in the external surface of the body and in fluid communication with the fluid passage within the body.

2. The hub of claim 1, wherein the fluid passage comprises a first conduit extending from the fluid inlet to a fluid outlet at the proximal end of the body, and a second fluid conduit extending from the first fluid conduit to the biosensor receiving receptacle and in fluid communication with the first conduit.

3. The hub of claim 1, wherein the fluid passage comprises a conduit extending from the fluid inlet to a fluid outlet at the proximal end of the body, and a fluid chamber in fluid communication with the fluid conduit and the biosensor receiving receptacle.

4. (canceled)

5. The hub of claim 1, wherein the recess comprises a flat area for receiving the biosensor.

6. The hub of claim 5, wherein the recess further comprises upstanding walls surrounding the flat area.

7. (canceled)

8. The hub of claim 1, further comprising one or more electrical contacts disposed on the external surface of the body for electrical communication with a biosensor disposed in the biosensor receiving receptacle.

9. The hub of claim 8, wherein the one or more electrical contacts are disposed adjacent the biosensor receiving receptacle.

10. The hub of claim 8, wherein the one or more electrical contacts are configured for electrical communication with a biomarker reader.

11. The hub of claim 1, wherein the body is configured for insertion into a biomarker reader with a biosensor disposed within the biosensor receiving receptacle.

12. The hub of claim 1, further comprising a needle or a tube mounted to the fluid inlet of the body.

13. The hub of claim 1, wherein the body is configured to mount to a device for fluid collection.

14. (canceled)

15. The hub of claim 1, wherein the body is configured to mount to a syringe, a sample collection tube, or a vacuum collection tube.

16. The hub of claim 1, further comprising a biosensor disposed within the biosensor receiving receptacle.

17. The hub of claim 16, further comprising a covering disposed over the biosensor.

18. The hub of claim 17, wherein the covering comprises a removable tab, the biosensor affixed to the tab for removal with the tab from the hub.

19. The hub of claim 18, wherein the tab includes an adhesive material on one surface, the biosensor affixed to the tab with the adhesive material.

20. The hub of claim 19, wherein the tab includes one or more regions extending beyond edges of the biosensor, and the adhesive material is further affixed to the external surface of the body at the one or more regions extending beyond the edges of the biosensor.

21. The hub of claim 16, wherein the biosensor further includes one or more electrical contacts configured for electrical communication with a biomarker reader.

22. The hub of claim 16, wherein the biosensor comprises at least one biosensor active area disposed in fluid communication with the fluid passage within the hub, the biosensor active area comprising a plurality of nanoelements disposed on a nanosubstrate, each nanoelement functionalized with an antibody or antigen binding fragment thereof, wherein fluid in the fluid passage within the body contacts the at least one biosensor active area of the biosensor.

23. The hub of claim 22, wherein the nanoelements comprise nanoparticles, nanotubes, nanocrystals, dendrimers, or nanowires.

24. The hub of claim 22, wherein the nanoelements comprise polystyrene, poly(lactic-co-glycolic acid), carbon nanotubes, single walled carbon nanotubes, organic nanotubes, polystyrene latex nanoparticles, silica nanoparticles, or proteins.

25. The hub of claim 22, wherein the plurality of nanoelements comprises different types of nanoelements, each type functionalized with a different type of antibody or antigen binding fragment thereof.

26. The hub of claim 22, wherein the biosensor active area comprises a plurality of regions, each region including a subset of the nanoelements, the nanoelements within each subset functionalized with a different type of antibody or antigen binding fragment thereof.

27.-70. (canceled)

71. A method of diagnosing a disease or disorder associated with a biomarker in a subject, the method comprising:
collecting a biological sample from the subject using an immunosensing device, the immunosensing device comprising the hub of claim 1 and a biosensor mounted to the hub;
contacting the biosensor the biological sample; and
determining the presence or absence of the biomarker within the biological sample.

72.-85. (canceled)
